(19) United States
(12) Patent Application Publication
(10) Pub. No.: US 2025/0356973 A1
(43) Pub. Date: Nov. 20, 2025

(54) LLM TIME SERIES ANALYSIS FOR MEDICAL DECISION MAKING

(71) Applicant: NEC Laboratories America, Inc., Princeton, NJ (US)

(72) Inventors: Wei Cheng, Princeton Junction, NJ (US); Wenchao Yu, Plainsboro, NJ (US); Haifeng Chen, West Windsor, NJ (US)

(21) Appl. No.: 19/206,304

(22) Filed: May 13, 2025

Related U.S. Application Data
(60) Provisional application No. 63/647,196, filed on May 14, 2024.
(60) Provisional application No. 63/650,021, filed on May 21, 2024.

(51) Int. Cl. G16H 20/00; G16H 10/60; G16H 50/20

(52) U.S. Cl. CPC G16H 20/00; G16H 10/60; G16H 50/20

(57) ABSTRACT

Methods and systems for time series analysis include encoding input time series data using a pre-trained encoder. The encoded time series is mapped to a format suitable for a large language model (LLM) using an alignment model. The mapped, encoded time series is analyzed using the LLM to generate a text output. An action is performed responsive to the text output.

### RELATED APPLICATION INFORMATION

This application claims priority to U.S. Patent Application No. 63/647,196, filed on May 14, 2024, and to U.S. Patent Application No. 63/650,021, filed on May 21, 2024, each incorporated herein by reference in its entirety.

### BACKGROUND

### Technical Field

The present invention relates to time series analysis and, more particularly, to the use of large language models (LLMs) for analyzing time series date.

### Description of the Related Art

LLMs can be used to interpret and summarize large volumes of input data, particularly for tasks that use natural language outputs like classification and captioning. In some cases the LLM can be used to predict future time series information based on a past sequence. However, operating in the natural language domain limits the applicability of LLMs, making them difficult to use in non-linguistic domains.

### SUMMARY

A method for time series analysis includes encoding input time series data using a pre-trained encoder. The encoded time series is mapped to a format suitable for a large language model (LLM) using an alignment model. The mapped, encoded time series is analyzed using the LLM to generate a text output. An action is performed responsive to the text output.

A system for time series analysis includes a hardware processor and a memory that stores a computer program. When executed by the hardware processor, the computer program causes the hardware processor to encode input time series data using a pre-trained encoder, to map the encoded time series to a format suitable for a large language model (LLM) using an alignment model, to analyze the mapped, encoded time series using the LLM to generate a text output, and to perform an action responsive to the text output.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a system for analyzing time series data using a large language model (LLM) where an alignment model maps the output of a pretrained time series encoder to a format suitable for the LLM, in accordance with an embodiment of the present invention;

FIG. 2 is a block/flow diagram of a method for analyzing and responding to time series data using an LLM, in accordance with an embodiment of the present invention;

FIG. 3 is a block diagram of a healthcare facility where time series analysis of patients' medical data is performed using an LLM, in accordance with an embodiment of the present invention;

FIG. 4 is a block diagram of a computing system that can perform time series analysis using an LLM, in accordance with an embodiment of the present invention;

FIG. 5 is a diagram of an exemplary neural network architecture that can be used to implement part of an alignment model, in accordance with an embodiment of the present invention; and

FIG. 6 is a diagram of an exemplary deep neural network architecture that can be used to implement part of an alignment model, in accordance with an embodiment of the present invention.

### DETAILED DESCRIPTION OF PREFERRED EMBODIMENTS

A self-supervised alignment model can be used to map time series data onto word embeddings, thereby making it easier for a large language model (LLM) to handle such inputs. The self-supervised alignment model may enhance the LLM's comprehension of time series information by aligning time series inputs with word embeddings. The LLM can thereby perform linguistic tasks, such as captioning and classification, on purely numerical time series inputs.

The self-supervised alignment model needs no labeled data, instead using self-supervision to align the time series data with language representations. Mapping the time series data to word embeddings translates the numerical time series data into a format that is compatible with word embeddings. These features empower an LLM to perform tasks that generate relevant natural language text, thereby enhancing the model's adaptability to applications involving time series data.

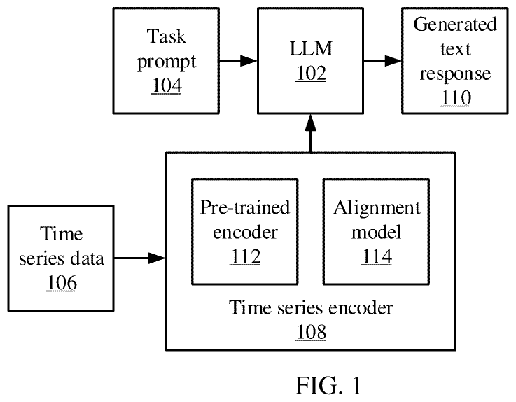

Referring now to FIG. 1, a time series analysis system is shown. The system includes an LLM 102, which performs a task that is set by a task prompt 104. For example, the text prompt 104 may instruct the LLM 102 to perform classification or captioning, thereby generating a text response 110. The LLM 102 itself may be an appropriate pre-trained language model, with or without domain-specific fine-tuning. An example of classification may include anomaly detection, indicating whether the input time series data 106 represents normal or abnormal behavior of a source system. An example of captioning may include generating text that describes the input time series data 106. Such captioning may include a summarization of the information conveyed by the input time series data 106.

Time series encoder 108 accepts the input time series data 106, for example as raw numerical and/or categorical values. The time series encoder 108 generates a representation of the input time series data 106 that can be processed by the LLM 102, for example by aligning the input time series data 106 with a natural language embedding.

The time series data 106 can include any appropriate type of information that is distributed across a period of time. For example, the time series data 106 may come from one or more sensors, each making respective measurements. The measurements may be made at fixed or variable intervals, or may be prompted by external stimuli. The measurements may furthermore be numerical information or categorical information. An example of numerical sensor information may include temperature values from a temperature sensor, while an example of categorical information may include a system's operational state.

In some embodiments, the time series data 106 may represent medical information relating to a patient. For example, the time series data 106 may include sensor measurements relating to the patient's physical condition, such as heart rate, blood pressure, and blood oxygen saturation measurements. The time series data 106 may further include categorical measurements, such as the patient's state of consciousness. In such a context, the generated text response 110 may include a diagnosis of the patient's health condition, a prediction of the patient's prognosis, and a treatment recommendation.

In some embodiments, the time series encoder 108 may include a pre-trained time series encoder 112 and an alignment model 114. The pre-trained encoder 112 may be used to process the raw input time series data 106 and may be implemented using, e.g., a temporal convolutional network or transformer-based architecture. The pre-trained encoder 112 generates fixed-dimensional encodings that capture the temporal patterns and features of the input time series data 106.

The alignment model 114 transforms the fixed-dimensional time series encodings into a format that is compatible with the LLM's language space. The alignment model 114 may be implemented using any appropriate neural network architecture, such as a feed-forward network or a network with an attention mechanism. The alignment model 114 learns a mapping that preserves the semantic meaning of the time series data while making it interpretable by the LLM 102. Thus the alignment model 114 accepts a vector embedding as input and outputs a vector in the space of the LLM 102.

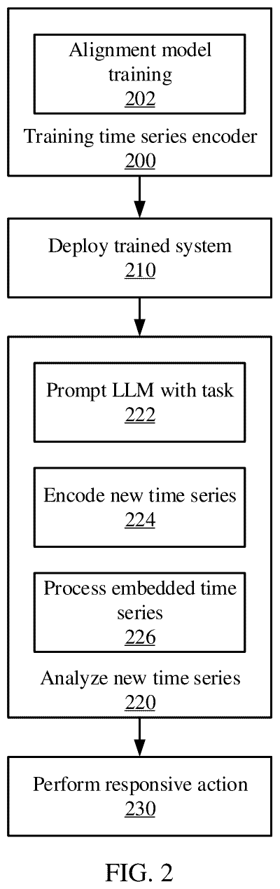

Referring now to FIG. 2, a method of training and using an LLM 102 with a time series encoder 108 is shown. Block 200 trains the time series encoder 108, for example using alignment model training 202. The alignment model training 202 operates on the parameters of the alignment model 114, without affecting the parameters of the LLM 102 or the pre-trained encoder 112. The alignment model training 202 may be performed using a contrastive loss, comparing the output of the LLM 102 (e.g., a classification) as compared to an expected output (e.g., a label provided for an instance of time series data). Backpropagation may be used to update the parameters of the time series encoder 108 in accordance with the loss.

Given a lack of paired time-series sequences with associated textual data, the training 202 of the alignment model 114 may be performed in a self-supervised manner. The self-supervised training includes generating prompts based on a set of training time series data. Each task instance may include a query time series for the LLM 102, a series of answer candidates, with each answer being a candidate time series that is indexed, converting the task to a multiple-choice classification. The correct answer index may be expressed in natural language.

The self-supervised training task may include time series denoising. To construct a task instance, a target time series or fraction thereof may be sampled from a training time series dataset. Noise may be added to the sample to generate a query time series. There may also be K-1 time series samples or fractions thereof other than the target time series, to use as contrastive answers. The contrastive answers are shuffled with the target time series to generate the answer candidates, with the index of the target time series being selected as the correct answer. Adding and removing noise makes the alignment model 114 more robust.

The training 202 may use a training objective that maximizes the likelihood of the correct answer, given the prompts. This may include a loss function that measures the difference between the LLM's generated responses and the correct answers. The alignment model 114 is trained to minimize this loss, thereby learning to map the time series encodings output by the pre-trained encoder 112 to the LLM's language space.

The loss function may be formulated as the negative log-likelihood of the correct answers. Given a prompt P and a correct answer A, the likelihood L may be expressed as:

$$L = log p ( A \vert P , E )$$

where E represents the time series encoding from the pre-trained encoder 112, p (·) indicates a probability function, and A|P denotes that P is appended to A. The alignment model 114 is trained to maximize L across all instances in the training set. The training process thus iterates over a large number of time series instances, generating prompts and answers, adjusting the parameters of the alignment model 114 to maximize the likelihood of correct answers.

Once the times series encoder 108 has been trained, block 210 deploys the trained system. This deployment may include transferring the time series encoder 108 and the LLM 102 to a computer system where new time series information is available, such as at a healthcare facility. In some cases, where the training 200 is performed at the same location as the analysis of new time series data 220, the deployment 210 may be omitted.

Block 220 uses the trained time series encoder 108 to analyze new time series data 106, for example having been collected by one or more sensors. The analysis includes prompting the LLM 102 with the task prompt 104, thereby providing the LLM 102 with instructions as to the type of analysis that is to be performed. Block 224 encodes the new time series data 106 using the time series encoder 108 to generate an embedded time series. Block 226 then uses the embedded time series as input to the LLM 102, which uses it to generate text response 110.

Block 230 then performs an action responsive to the generated text response 110. Following the example of time series data that tracks a patient's health information, the responsive action may include generating a medical alert if the patient's condition deteriorates. Another example may be to automatically administer a treatment to the patient, for example by automatically administering a medicating responsive to a diagnosis and recommended treatment determined by the LLM 102.

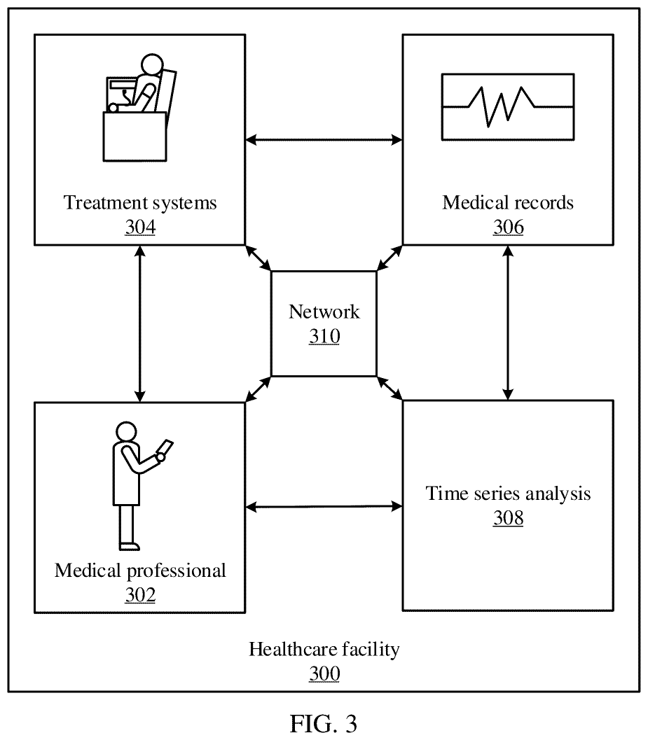

Referring now to FIG. 3, a diagram of time series analysis is shown in the context of a healthcare facility 300. Time series analysis 308 may be performed using an LLM, for example by encoding new time series data and performing an alignment that maps the time series data to a format that the LLM can handle. This time series analysis 308 may operate on medical information gathered regarding a patient's health condition, including information from sensors and/or medical records 306.

The healthcare facility may include one or more medical professionals 302 who review information extracted from a patient's medical records 306 to determine their healthcare and treatment needs. These medical records 306 may include self-reported information from the patient, test results, and notes by healthcare personnel made to the patient's file. Treatment systems 304 may furthermore monitor patient status to generate medical records 306 and may be designed to automatically administer and adjust treatments as needed.

Based on information drawn from the time series analysis 308, the medical professionals 302 may then make medical decisions about patient healthcare suited to the patient's needs. For example, the medical professionals 302 may make a diagnosis of the patient's health condition and may prescribe particular medications, surgeries, and/or therapies.

The different elements of the healthcare facility 300 may communicate with one another via a network 310, for example using any appropriate wired or wireless communications protocol and medium. Thus time series analysis 308 receives data from treatment systems 304, medical professionals 302, and from medical records 306, and updates the medical records 306 with the generated text output of the LLM. The time series analysis 308 may further coordinate with treatment systems 304 in some cases to automatically administer or alter a treatment. For example, generated text output a dangerous health condition, the treatment systems 304 may automatically alter or halt the administration of the treatment.

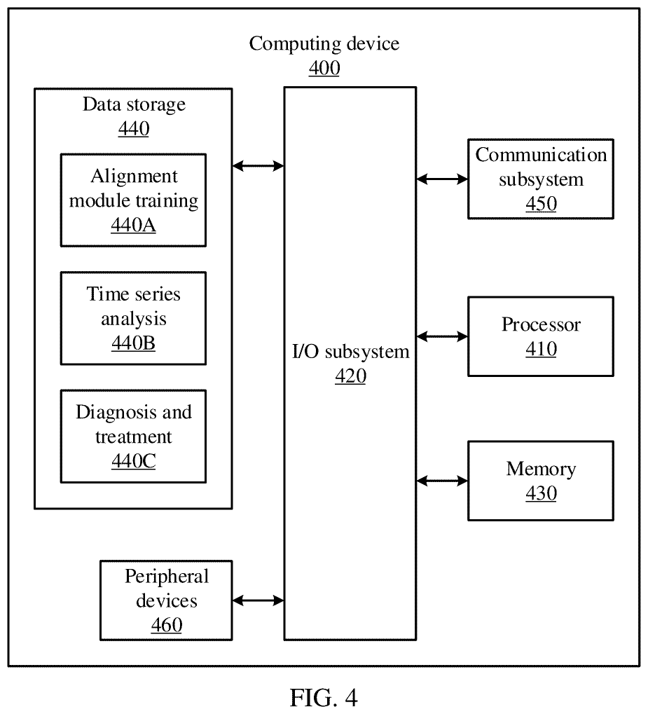

Referring now to FIG. 4, an exemplary computing device 400 is shown, in accordance with an embodiment of the present invention. The computing device 400 is configured to perform visual question answering.

The computing device 400 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 400 may be embodied as one or more compute sleds, memory sleds, or other racks, sleds, computing chassis, or other components of a physically disaggregated computing device.

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for training an alignment model, 440B for performing time series analysis, and/or 440C for performing diagnosis and treatment. Any or all of these program code blocks may be included in a given computing system. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

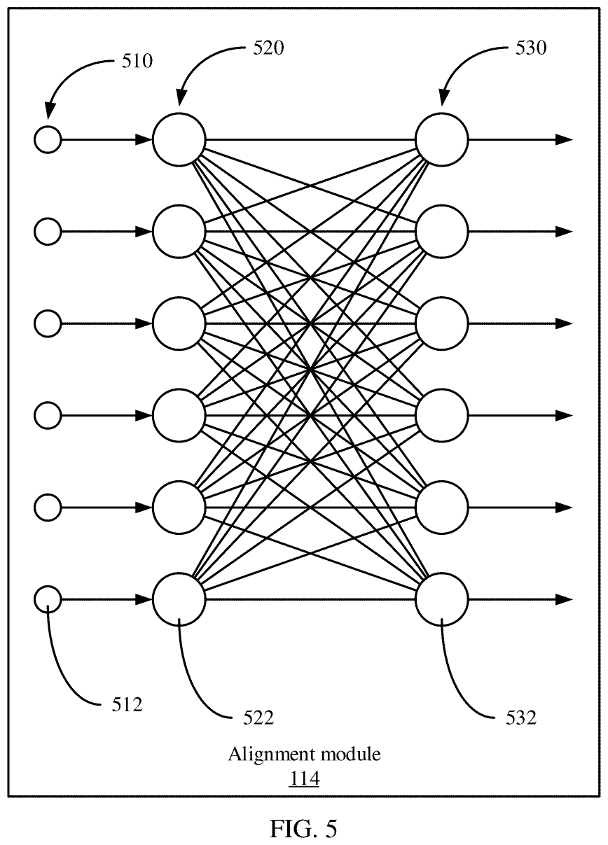

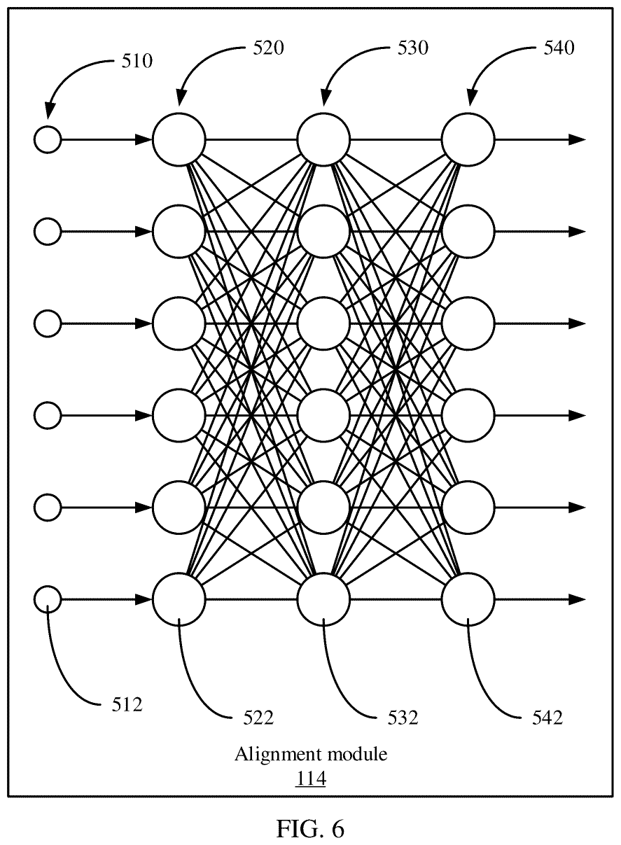

Referring now to FIGS. 5 and 6, exemplary neural network architectures are shown, which may be used to implement parts of the present machine learning models, such as the alignment model 114. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 532 in the one or more computation (hidden) layer(s) 530 perform a nonlinear transformation on the input data 512 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for time series analysis, comprising:
encoding input time series data using a pre-trained encoder;
mapping the encoded time series to a format suitable for a large language model (LLM) using an alignment model;
analyzing the mapped, encoded time series using the LLM to generate a text output; and
performing an action responsive to the text output.

2. The method of claim 1, further comprising training the alignment model using a self-supervised training process.

3. The method of claim 2, wherein the self-supervised training process includes adding noise to a training time series and training the alignment model to identify the training time series.

4. The method of claim 3, wherein training the alignment model to identify the training time series includes a selection between the training time series and at least one contrastive time series sample.

5. The method of claim 4, wherein training the alignment model includes maximizing a negative log-likelihood of selecting the training time series.

6. The method of claim 1, wherein analyzing the mapped, encoded time series further includes adding a prompt that specifies a task for the LLM to perform.

7. The method of claim 1, wherein the input time series includes measurements taken of a patient's medical state.

8. The method of claim 7, wherein the action includes changing or halting a treatment to the patient.

9. The method of claim 7, wherein the text output is used to assist in medical decision making.

10. The method of claim 1, wherein the alignment model is implemented as a machine learning model.

11. A system for time series analysis, comprising:
a hardware processor; and
a memory that stores a computer program which, when executed by the hardware processor, causes the hardware processor to:
encode input time series data using a pre-trained encoder;
map the encoded time series to a format suitable for a large language model (LLM) using an alignment model;
analyze the mapped, encoded time series using the LLM to generate a text output; and
perform an action responsive to the text output.

12. The system of claim 11, wherein the computer program further causes the hardware processor to train the alignment model using a self-supervised training process.

13. The system of claim 12, wherein the self-supervised training process includes addition of noise to a training time series and training the alignment model to identify the training time series.

14. The system of claim 13, wherein the computer program further causes the hardware processor to select between the training time series and at least one contrastive time series sample.

15. The system of claim 14, wherein the computer program further causes the hardware processor to maximize a negative log-likelihood of selection of the training time series.

16. The system of claim 11, wherein analysis of the mapped, encoded time series further includes addition of a prompt that specifies a task for the LLM to perform.

17. The system of claim 11, wherein the input time series includes measurements taken of a patient's medical state.

18. The system of claim 17, wherein the action includes changing or halting a treatment to the patient.

19. The system of claim 17, wherein the text output is used to assist in medical decision making.

20. The system of claim 11, wherein the alignment model is implemented as a machine learning model.
